(19) United States
(12) Patent Application Publication
(10) Pub. No.: US 2017/0312380 A1
(43) Pub. Date: Nov. 2, 2017

(54) SCENT DISPENSER AND SCENT CELL THEREFOR

(71) Applicant: NOVIA PRODUCTS LLC, Portland, ME (US)

(72) Inventors: William Webster, Portland, ME (US); Randy M. Oliver, Limerick, ME (US); Timothy Thomson, Greensboro, NC (US); David Gallant, Newfield, ME (US)

(73) Assignee: NOVIA PRODUCTS LLC, Portland, ME (US)

(21) Appl. No.: 15/499,054

(22) Filed: Apr. 27, 2017

Related U.S. Application Data
(60) Provisional application No. 62/328,760, filed on Apr. 28, 2016.

(51) Int. Cl. A61L 9/012; B05B 7/24

(52) U.S. Cl. CPC A61L 9/012; B05B 7/2464; A61L 2209/133

(57) ABSTRACT

A scent dispenser is provided which comprises a cylinder having a cylinder chamber for receiving a hollow piston which includes a piston body that is closely fitted to the cylinder chamber and is slideable therein, upward to a fully extended position and downward to a fully compressed position. The piston body also includes a cylindrical piston chamber having an opening at a bottom end of the piston body, which body is sized and shaped to receive a scent cell therein. The piston further includes a cover attached to or integral with an upper end of the piston body. The scent cell may be in the form of a substrate of nonwoven fibrous material capable of allowing air to flow readily through the scent cell and through a volatile scented substance contained therein. The scent cell may further comprise a coating material capable of reversibly absorbing or adsorbing the volatile scented substance, while allowing air to flow readily through the scent cell. The coating material covers or coats the substrate and may comprise a substantially hydrophilic foam.

### CROSS-REFERENCE TO RELATED APPLICATIONS

The present application claims the benefit of U.S. Provisional Patent Application No. 62/328,760, filed on Apr. 28, 2016, the disclosure of which is incorporated by reference herein.

### FIELD OF THE INVENTION

The disclosed subject matter relates to dispensers of scents into the environment and/or absorbers of odors or materials from the environment.

### BACKGROUND OF THE INVENTION

Many forms of dispensers of scents into the environment, such as the household or other building interior environment, or devices for removing odors or materials from the environment, are known in the art. Applicants have improved the performance of such devices while maintaining simplicity and economics of manufacture and operation.

### SUMMARY OF THE INVENTION

According to one exemplary embodiment, a scent dispenser comprises a cylinder having a cylinder chamber for receiving a hollow piston. The piston includes a piston body that is closely fitted to the cylinder chamber and is slideable therein, upward to a fully extended position and downward to a fully compressed position. The piston body includes a cylindrical piston chamber having an opening at a bottom end of the piston body and is adapted for receiving a scent cell therein. According to the exemplary embodiment the piston further includes a cover attached to or integral with an upper end of the piston body.

According to an exemplary embodiment, the piston body may also have a ventilation port proximate its top end for allowing air to flow into or out of the piston chamber when the piston body is in the cylinder chamber. An arrangement of a lock pin on the cylinder and an L-shaped groove on the piston body provide means for releasing the piston for upward movement and for locking the piston in place when the piston body is in its fully compressed position in the cylinder chamber. Motive force for the upward movement of the piston toward the locking position is provided by a spring positioned in the cylinder chamber and in contact with the bottom end of the piston body.

According to one exemplary embodiment, a scent cell comprises: a substrate comprising a nonwoven fibrous material and being capable of allowing air to flow readily through the scent cell; a volatile scented substance; and, optionally, a coating material capable of reversibly absorbing or adsorbing the volatile scented substance and capable of allowing air to flow readily through the scent cell, wherein the coating material comprises a substantially hydrophilic foam and covers or coats the substrate. Interstitial spaces within the nonwoven fibrous material form air passages penetrating through the scent cell.

In another exemplary embodiment, the aforesaid scent dispenser includes the aforesaid scent cell.

During operation of the scent dispenser, upward movement of the piston causes suction to form in the cylinder chamber, drawing air into the cylinder chamber through the ventilation port and the piston chamber. When a scent cell is positioned in the piston chamber, a portion of the air passes through the scent cell, carrying some of the scented substance with it. Downward movement of the piston compresses the scented air in the cylinder chamber, forcing it through the piston chamber to be expelled through the ventilation port. A portion of the air from the cylinder chamber again passes through the scent cell, carrying away more of the scented material to be expelled through the ventilation port with the air.

### BRIEF DESCRIPTION OF THE DRAWINGS

For a better understanding of the present invention, reference is made to the following detailed description of the exemplary embodiments considered in conjunction with the accompanying drawings, in which like structures are referred to by like numerals throughout the several views, and in which:

FIG. 1 is a perspective view of a scent dispenser according to an embodiment of the present invention;

FIG. 2 is an exploded perspective view of the scent dispenser of FIG. 1;

FIG. 3 is a front elevational view of the piston of the scent dispenser of FIG. 1;

FIG. 4 is a right elevational view of the piston of FIG. 3;

FIG. 5 is a right elevational view of a cross-section of the piston of FIG. 3, taken through a vertical plane through the piston;

FIG. 6 is a top plan view of the piston of FIG. 3;

FIG. 7 is a front elevational view of the cylinder of the scent dispenser of FIG. 1;

FIG. 8 is a left elevational view of a cross-section of the cylinder of FIG. 7, taken at a vertical plane through the cylinder;

FIG. 9 is a top plan view of the cylinder of FIG. 7;

FIG. 10 is a top plan view of the door of the scent dispenser of FIG. 1;

FIG. 11 is a left elevational view of a cross-section of the door of FIG. 10, taken at a vertical plane through a fingernail slot recessed into the door;

FIG. 12 is a rear view of the door of FIG. 10, oriented such that the top of the door is at the left of the figure;

FIG. 13 is a side view of a scent dispenser according to another embodiment;

FIG. 14 is a cross-section view of the scent dispenser of FIG. 13 taken along the lines 14-14 in FIG. 13;

FIG. 14A is a close-up view of area 14A in FIG. 14;

FIG. 14B is a close-up view of area 14B in FIG. 14;

FIG. 15 is a top view of the scent dispenser of FIG. 13;

FIG. 16 is an exploded perspective view of another exemplary embodiment of a scent dispenser;

FIG. 17 is a top plan view of the scent dispenser of FIG. 16 in its assembled state;

FIG. 18 is a left elevational view of a cross-section of the scent dispenser of FIGS. 16 and 17 in its assembled state, taken along the lines 13-13 in FIG. 17, and showing the piston in its fully extended position;

FIG. 19 is a left elevational view of a cross-section of the scent dispenser of FIGS. 16 and 17 in its assembled state, taken along the lines 13-13 in FIG. 17, and showing the piston in its fully compressed position; and

FIG. 20 is a front perspective view of the piston of the scent dispenser of FIGS. 16-19.

### DETAILED DESCRIPTION OF THE INVENTION

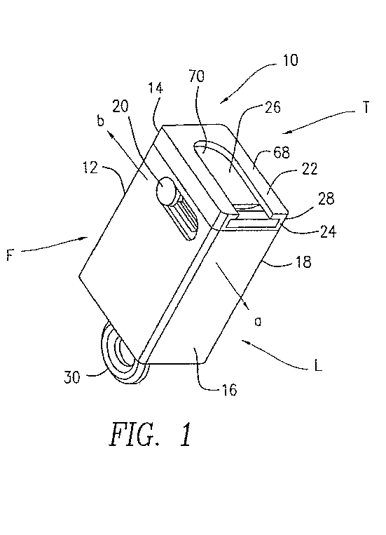

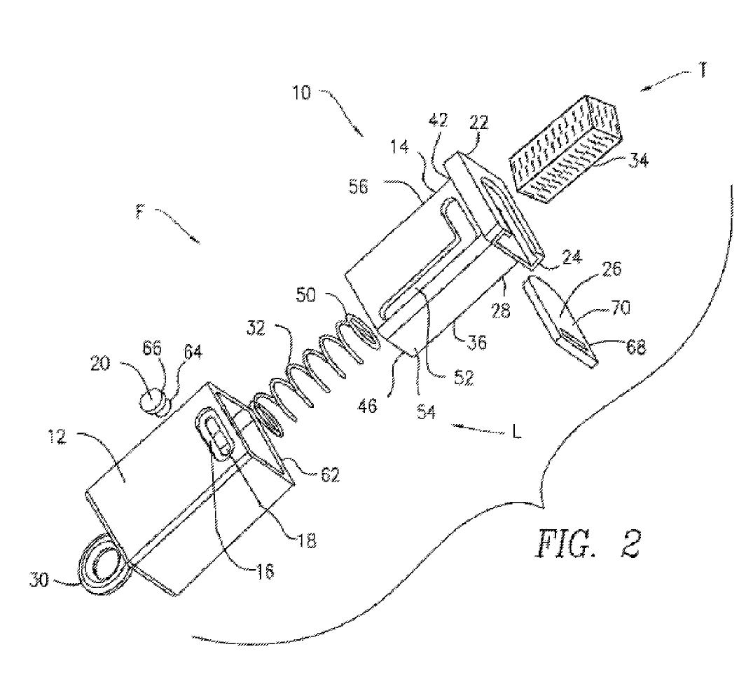

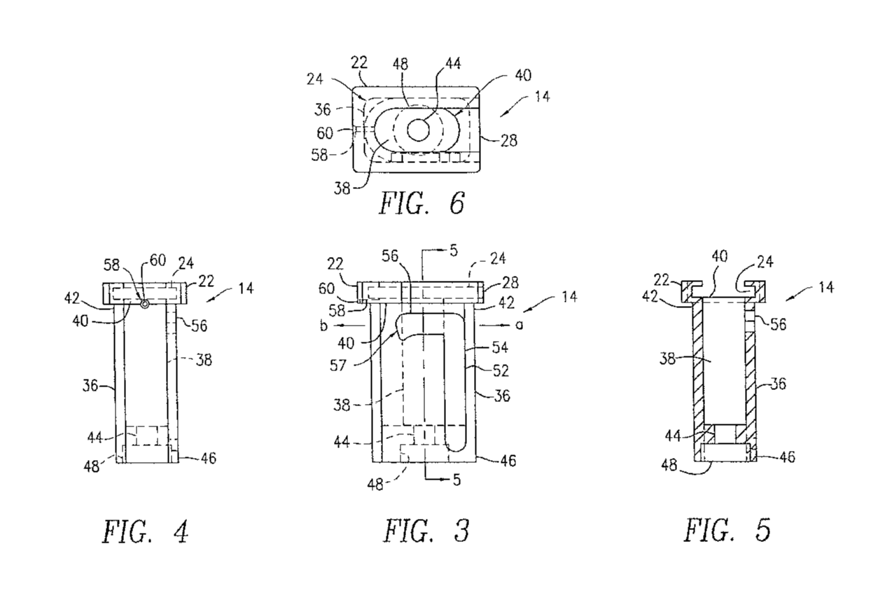

FIGS. 1-12 present views of a scent dispenser 10 and its component parts according to various exemplary embodiments. FIGS. 1 and 2 are perspective views of the scent dispenser 10 that illustrate relationships among various elements of the scent dispenser 10. However, consistent with the nature of perspective views, some elements of the scent dispenser 10 discussed herein are not visible in those figures. All such elements of the scent dispenser 10 are discussed further in relation to FIGS. 3-12, which present elevational, plan or cross-sectional views of certain component parts of the scent dispenser 10. Terms indicating position, orientation or direction of motion are used throughout the discussion of FIGS. 1-12 in relation to the component parts and are consistent with the orientation of the parts shown in FIGS. 1 and 2 unless otherwise expressly noted, or by default are in relation to the orientation within the illustration of the FIG. on the page. Such terms are used for the purpose of facilitating discussion, and not to limit the embodiments to the particular ones shown and described or to limit physical orientation in actual use to any particular coordinate system, such as horizontal, vertical and front, back and side while the device is in actual use.

FIG. 1 is a perspective view of the scent dispenser 10 in a fully-assembled and closed state. The scent dispenser 10 is oriented such that it may be viewed from the top direction “T”, the front direction “F”, and the left side direction “L”. In regard to the direction of motion, “leftward” is the direction toward the left side of the scent dispenser 10 (i.e., the side indicated by “L”) and is indicated by the arrow labeled “a”. “Rightward” is the direction that is opposite to the leftward direction, and is indicated by the arrow labeled “b”. The scent dispenser 10 includes a cylinder 12 arranged to receive a piston 14 such that the piston 14 may move upward or downward within the cylinder 12. The cylinder 12 defines a recessed ledge 16 and a slot 18, which are visible at the front of the scent dispenser 10. A lock button 20 resides in the slot 18 such that the lock button 20 may slide leftward or rightward within the slot 18. The lock button 20 will be discussed further below in the present application.

Continuing to refer to FIG. 1, the piston 14 has a U-shaped flange 22, visible at the top of the scent dispenser 10, as seen more clearly in FIGS. 2 and 5 to possess such a U-shape in side view, which flange 22 defines a groove 24 that is arranged to receive a door 26 through the open end 28 of the “U” such that the door 26 fits closely within the groove 24 but may be moved leftward or rightward within the groove 24. In some embodiments, such as the illustrated embodiment, the piston 14 is arranged such that the U-shaped flange 22 contacts the cylinder 12 when the piston 14 is received to its maximum depth within the cylinder 12. A ring 30 that is attached to or integral with the cylinder 12 may optionally be provided, e.g. for the attachment of the dispenser 10 to another object, such as with a lanyard (not shown).

FIG. 2 is an exploded perspective view of the scent dispenser 10 of FIG. 1, showing additional elements of the scent dispenser 10 that are not visible in FIG. 1. The scent dispenser 10 of FIG. 2 is oriented such that it may be viewed from the top direction “T”, the front direction “F”, and the left side direction “L”. As seen in FIG. 2, in addition to the elements discussed with respect to FIG. 1, the scent dispenser 10 includes a spring 32 and a scent cell 34. The spring 32 and scent cell 34 will be discussed further below in the present application.

FIGS. 3-6 present elevational, plan and cross-sectional views of the piston 14, of which FIG. 3 is a front elevational view, FIG. 4 is a right elevational view, FIG. 5 is a right elevational cross-sectional view, taken through a vertical plane through the piston 14, along lines 5-5 shown in FIG. 4, and FIG. 6 is a top plan view. Terms indicating position, orientation or direction of motion of the piston 14 or its elements are defined in relation to the front elevational view of the piston 14 in FIG. 3, and thus are consistent with their usage with respect to FIGS. 1 and 2. In regard to the direction of motion, “leftward” is the direction toward the left side of the scent dispenser 10 (i.e., the side indicated by “L”) and is indicated by the arrow labeled “a”. “Rightward” is the direction that is opposite to the leftward direction, and is indicated by the arrow labeled “b”. To understand the elements and function of the piston 14, FIGS. 3-6 should be read together and in conjunction with FIGS. 1 and 2.

Referring to FIGS. 3-6 in conjunction with FIGS. 1 and 2, the piston 14 has a cylindrical piston body 36, which is attached to or integral with the U-shaped flange 22. The piston body 36 is partially hollow and defines a piston chamber 38 arranged such that the scent cell 34 may be received entirely within the piston chamber 38. The piston body 36 also defines an opening 40 through the top end 42 of the piston body 36 which is arranged to allow passage of the scent cell 34 into or out of the piston chamber 38. The piston body 36 also defines an opening 44 at the bottom end 46 of the piston body 36 which is arranged to prevent passage of the scent cell 34 out of the piston chamber 38 while providing fluid communication between the piston chamber 38 and the environment of the piston body 36, i.e., into the cylinder chamber volume V (see FIG. 8) formed as the piston 14 is withdrawn from (i.e., moved upward in) the cylinder 12. In some embodiments, such as the illustrated embodiment, the piston body 36 also defines a receiving bore 48 at the bottom end 46 of the piston body 36 that is arranged to securely receive an end 50 of the spring 32. In some embodiments, such as the illustrated embodiment, the receiving bore 48 also provides fluid communication between the piston chamber 38 and the interior of the piston 12, through the opening 44.

Continuing to refer to FIGS. 3-6 in conjunction with FIGS. 1 and 2, the piston 14 is provided with an L-shaped groove 52 that is recessed into the piston body 36 and has a vertical leg 54 and a transverse leg 56. The L-shaped groove 52 is arranged such that its vertical leg 54 is oriented along the direction of travel of the piston 14 within the cylinder 12 and has a length equal to the distance over which the piston 14 is desired to travel. The L-shaped groove 52 is further arranged such that its transverse leg 56 is oriented to coincide with the slot 18 of the cylinder 12 when the piston body 36 is at its furthest limit of downward travel within the cylinder 12. In some embodiments, such as the illustrated embodiment, the transverse leg 56 has a notch 57 at its end furthest from the vertical leg 54. The notch 57 is arranged to receive the lock button 20 such that pressure must be applied to the lock button 20 to dislodge it from the notch 57. The L-shaped groove 52 is further arranged to further receive the lock button 20 in a manner that is discussed below in the present application.

Continuing to refer to FIGS. 3-6 in conjunction with FIGS. 1 and 2, the U-shaped flange 22 is arranged such as to completely expose the opening 40. The U-shaped flange 22 and the groove 24 are further arranged such that the door 26 may fit snugly within the groove 24 such as to completely cover the opening 40, thus preventing air flow through the opening 40. The U-shaped flange 22 is further arranged such that it is larger in at least one horizontal direction than the top end 42 of the piston body 36. In the illustrated embodiment, the U-shaped flange 22 is larger than the top end 42 in all horizontal directions.

Continuing to refer to FIGS. 3-6 in conjunction with FIGS. 1 and 2, the piston body 36 and U-shaped flange 22 define a ventilation port 58 that provides fluid communication between the piston chamber 38 and the ambient environment of the scent dispenser 10. The ventilation port 58 is positioned below the groove 24 such that the passage of air through the ventilation port 58 is not impeded by the door 26 when the door 26 is fully inserted into the U-shaped flange 22. In some embodiments, such as the illustrated embodiment, the ventilation port 58 is defined by the piston body 36 and the U-shaped flange 22 such that fluid communication between the piston chamber 38 and the ambient environment of the scent dispenser 10 is not blocked when the piston 14 is at its furthest limit of downward travel within the cylinder 12. In other embodiments, the ventilation port 58 may be entirely defined by the U-shaped flange 22. Other forms of ventilation opening may be envisioned, including one for within or partly by the door 26 in position to block most air flow into the piston chamber 38, but allowing smaller flow associated with a ventilation opening 58 as discussed above.

In some embodiments, such as the illustrated embodiment, the ventilation port 58 is formed opposite the open end 28 of the U-shaped flange. In other embodiments, the ventilation port 58 is formed at some other position along the U-shaped flange. In some embodiments, the ventilation port 58 has an effective diameter of 1 mm or less. In some embodiments, such as the illustrated embodiment, the ventilation port 58 is provided with a counterbore 60 that flares outward toward the ambient environment such as to reduce resistance of air flow into the piston chamber 38 through the ventilation port 58 and to aid the dispersal of scent passing out of the piston chamber 38 through the ventilation port 58. Air flow into and out of the piston chamber 38 occurs through the operation of the scent dispenser 10 as discussed below in the present application.

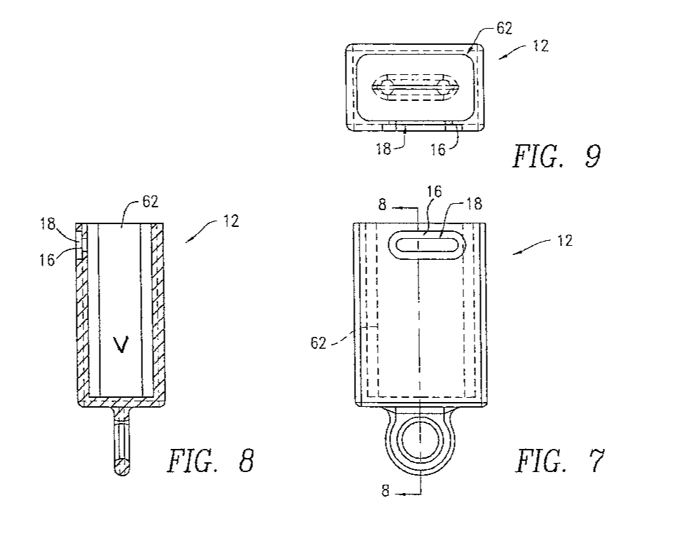

FIGS. 7-9 present elevational, plan and cross-sectional views of the cylinder 12, of which FIG. 7 is a front elevational view, FIG. 8 is a left elevational cross-sectional view taken at a vertical plane through the cylinder 12, along lines 8-8 in FIG. 7, and FIG. 9 is a top plan view. Terms indicating position, orientation or direction of motion of the cylinder 12 or its elements are defined in relation to the front elevational view of the cylinder 12 in FIG. 7, and thus are consistent with their usage with respect to FIGS. 1 and 2. To understand the elements and function of the cylinder 12, FIGS. 7-9 should be read together and in conjunction with FIGS. 1 and 2.

Referring to FIGS. 7-9 in conjunction with FIGS. 1 and 2, the cylinder 12 defines a cylindrical cylinder chamber 62 that is arranged to receive the piston body 36 such that the piston body 36 fits closely within the cylinder chamber 62 while allowing the piston body 36 to move readily upward or downward therein. The slot 18 penetrates the cylinder 12 such that the piston body 36 is exposed through the slot 18.

Referring to FIG. 2 in conjunction with FIGS. 3-7, the lock button 20 has a small end 64 that is arranged to reside in the slot 18 and a large end 66 that is arranged to reside on the recessed ledge 16. The lock button 20 is further arranged such that the small end 64 may extend through the slot 18 into the L-shaped groove 52 of the piston 14 when the large end 66 resides on the recessed ledge 16. In such an arrangement, the lock button 20 prevents the upward or downward movement of the piston 14 when the lock button 20 resides in the transverse leg 56 of the L-shaped groove 52 and allows the upward or downward movement of the piston 14 when it resides in the vertical leg 54 of the L-shaped groove 52. The slot 18 and L-shaped groove 52 are arranged such that, when the piston body 36 is at its furthest limit of downward travel into the cylinder 12, the lock button 20 may be moved from the vertical leg 54 to the transverse leg 56, or vice versa, by sliding the lock button 20 within the slot 18 leftward or rightward, as appropriate.

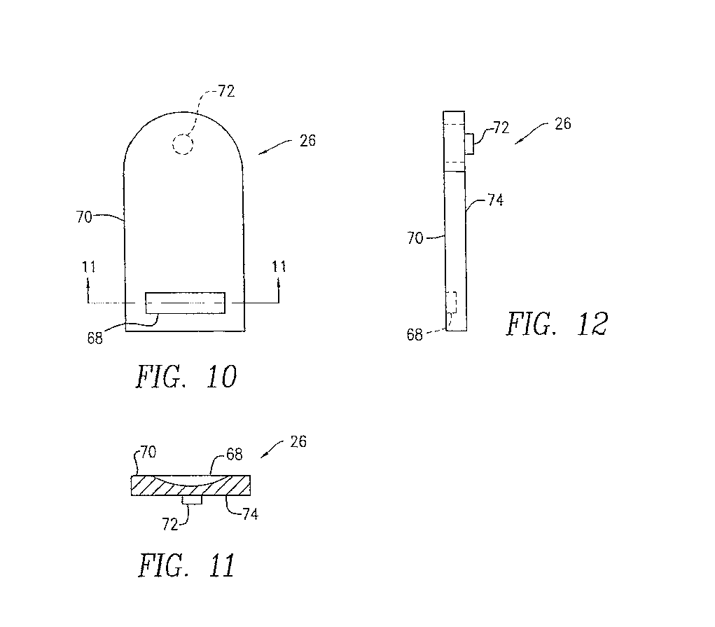

FIGS. 10-12 present elevational, plan and cross-sectional views of the door 26, of which FIG. 10 is a top plan view, FIG. 11 is a left elevational cross-sectional view taken at a vertical plane through a finger slot 68 recessed into a top surface 70 of the door 26, taken along lines 11-11 in FIG. 10, and FIG. 12 is a rear view, oriented such that the top surface 70 of the door 26 is at the left of FIG. 12. Terms indicating position, orientation or direction of motion of the door 26 or its elements are defined such as to be consistent with similar terms used in relation to FIGS. 1-6. To understand the elements and function of the door 26, FIGS. 10-12 should be read together and in conjunction with FIGS. 1-6.

Referring to FIGS. 10-12 in conjunction with FIGS. 1-6, in some embodiments, such as the illustrated embodiment, the door 26 is provided with a fingernail slot 68 recessed into a top surface 70 of the door 26, which may be used to conveniently move the door 26 rightward and leftward (as defined in the discussions of FIGS. 1 and 3) within the groove 24 of the U-shaped flange 22. In some embodiments, the fingernail slot 68 is arranged such as to conveniently receive the edge of a fingernail or of a small coin. The door 26 is also provided with a stub 72 that protrudes from a bottom surface 74 of the door 26. The stub 72 is arranged on the door 26 such that, with the door 26 within the groove 18 of the U-shaped flange 22, the stub 72 protrudes into the opening 40 of the piston chamber 38. The stub 72 protrudes into the opening 40 such that leftward movement of the door 26 is limited by contact between the stub 72 and the piston body 36 preventing the door 26 from being readily removed from the U-shaped flange 22. The stub 72 is further arranged on the door 26 such that, at the limit of its leftward movement, the door 26 exposes enough of the opening 40 that the scent cell 34 may readily pass through the opening 40 into or out of the piston chamber 38. In some embodiments, the stub 72 can be attached to a threaded member threaded engaging the door such that in one position the threaded member and stud are not extending beyond either side of the door 26. When the threaded portion is threaded into an opening shown in phantom in FIG. 12. In other embodiments, the stub 72 may be the end of a screw or tack entering the top surface 70 and protruding through the bottom surface 74.

Referring to FIG. 2 in conjunction with FIGS. 3-6, the spring 32 is arranged to fit within the cylinder chamber 62 in contact with bottom end 46 of the piston body 36. In some embodiments, such as the illustrated embodiment, the end 50 of the spring 32 is secured in the receiving bore 48 of the piston body 36 such as to center the spring 32 within the cylinder chamber 62. The spring 32 exerts an upward force on the piston 14 such that the piston 14 moves upward without being pulled. In some embodiments, such as the illustrated embodiment, the spring 32 is a helical compression spring.

Referring to FIG. 2, the scent cell 34 comprises a material that can absorb and/or adsorb a volatile scented substance and allows the ready passage of air through the scent cell 34. The scent cell 34 contains an amount of a volatile scented substance, such as those used in perfumes, for attracting game, as diet aids, for aroma therapy, for medical applications, or for other uses which are known or may become known. In some embodiments, the scent cell 34 is arranged such that the scented substance may be added directly to the scent cell 34 to replenish or change the scent.

The scent cell 34 may comprise any material that can carry and release volatile scented substances. In some embodiments of the invention, the scent cell 34 is made of an absorbent fibrous material having air passages penetrating therethrough. In some embodiments, for example, the scent cell 34 may comprise a nonwoven fibrous material substrate coated with a coating material, such as a substantially hydrophilic foam coating which is exposed at the surface and in interstitial spaces within the nonwoven fibrous material. The interstitial spaces within the nonwoven fibrous material form air passages penetrating through the scent cell 34 to allow air to flow through the scent cell 34. Suitable nonwoven fibrous materials include, for example without limitation, cotton, felt, silk, or combinations thereof. As will be recognized by persons of ordinary skill in the relevant art, such embodiments would be useful for example, when the volatile scented substances to be used to impart scent or odor to the scent cell 34 are of the types that may react with and degrade some hydrophobic foams which are sometimes used for the scent cell 34 (see, e.g., U.S. Pat. No. 8,544,766, which is incorporated herein by reference in its entirety). While any volatile scented substances known now or in the future may be used with scent cells 34 of this embodiment, scent cells 34 comprising nonwoven fibrous material will be particularly useful with certain volatile scented substances including for example, without limitation, cinnamon and citric essential oils.

While not intending to be limited, one possible embodiment of a process for producing scent cells 34 having nonwoven fibrous substrates coated with a coating material, such as a substantially hydrophilic foam, will now be described briefly. Where the substantially hydrophilic foam is produced by a process involving producing a prepolymer emulsion and then polymerizing or curing the emulsion, as will be understood by persons of ordinary skill in the relevant art, the scent cell 34 may be produced by contacting the substrate of nonwoven fibrous material with the prepolymer emulsion and then polymerizing or curing the emulsion. Contacting the substrate and prepolymer emulsion may be performed by any method known now or in the future by persons of ordinary skill in the relevant art, including without limitation, dipping or immersing the substrate in the prepolymer emulsion, or even by brushing, spraying or otherwise coating the emulsion onto the substrate. For example, without limitation, the substrate of nonwoven fibrous material may be provided as a sheet or block and then sprayed with the prepolymer emulsion, followed by polymerization or curing of the emulsion to form the substantially hydrophilic foam on the nonwoven fibrous substrate. The coated nonwoven material substrate is then cut into appropriately sized pieces to produce multiple scent cells 34, each of which fits into the piston chamber 38 of the piston 14. The scent cell 34 comprising a nonwoven fibrous material coated with a substantially hydrophilic foam may initially be any shape, such as for example, cylindrical or a block with a square or rectangular cross-sectional shape (see, e.g., FIG. 2). Regardless of shape, the scent cell 34 is press fit in to the piston chamber 38 of the piston 14 and held there by frictional forces.

The various elements of the scent dispenser 10 are arranged such that, when the scent dispenser 10 is fully assembled, the ventilation port 58 provides the only path by which air may move into or out of the scent dispenser 10. The fully-assembled scent dispenser 10 may be operated in the manner described herein with reference to FIGS. 1 and 2. Beginning in the closed state shown in FIG. 1, the lock button 20 is moved leftward (i.e., in the direction indicated by the arrow labeled “a” in FIGS. 1 and 3) within the slot 18 such that the small end 64 of the lock button 20 enters the vertical leg 54 of the L-shaped groove 52. This action frees the piston 14 to travel upward, with the motive force provided by the spring 32. As the piston 14 travels upward, suction is created within the cylinder chamber 62, drawing air into the confined cylinder chamber volume V formed as the piston 14 is withdrawn through the cylinder chamber 62, through the ventilation port 58 and the piston chamber 38. As the air is drawn in, some or all of the air passes through the scent cell 34, carrying some of the volatile scented material into the cylinder chamber 62.

After the piston 14 has traveled upward for some distance, it may be pressed downward into the cylinder 12, such as by a user applying pressure to the piston 14 to counteract the spring force of spring 32. Such action causes the air in the cylinder chamber 62 to become compressed, forcing air to pass from the cylinder chamber 62 through the piston chamber 38, to be expelled through the ventilation port 58. Some or all of the air again passes through the scent cell 34, carrying away an additional amount of the volatile scented material which is expelled with the air through the ventilation port 58. The further the travel of the piston 14, the more air, and scent, is expelled. When the piston 14 is fully inserted into the cylinder 12, the lock button 20 may be moved rightward (i.e., in the direction indicated by the arrow labeled “b” in FIGS. 1 and 3) into the transverse leg 56 of the L-shaped groove 52, preventing upward movement of the piston 14. Other mechanical or electromechanical mechanisms (not shown) may be utilized to move the piston 14 in the direction in which air is expelled from the cylinder chamber volume V returning through the scent cell 34 and out the ventilation opening 58.

During operation of the scent dispenser 10, the door 26 remains fully-inserted into the groove 24 of the U-shaped flange 22, thus blocking air flow through the opening 40 of the piston chamber 38. When the scent dispenser 10 is not being operated, the door 26 may be moved leftward (i.e., in the direction indicated by the arrow labeled “a” in FIGS. 1 and 3) to expose the opening 40 such that the scent cell 34 may be removed or replaced, or that scented material may be added to the scent cell 34.

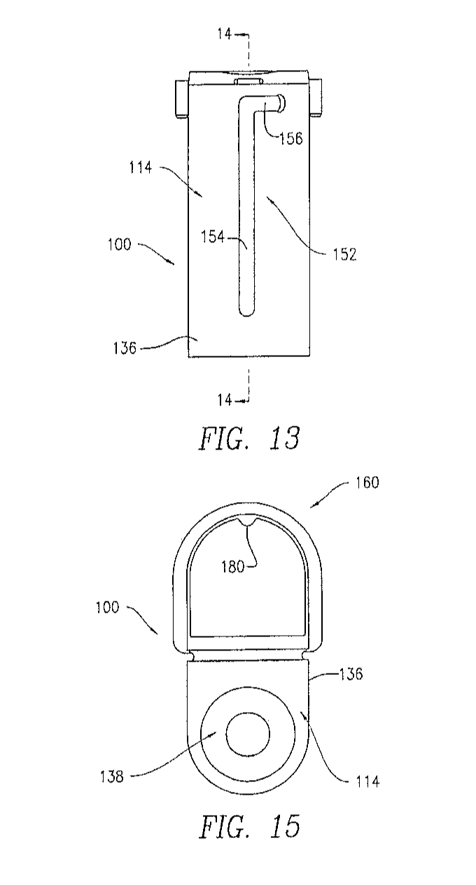

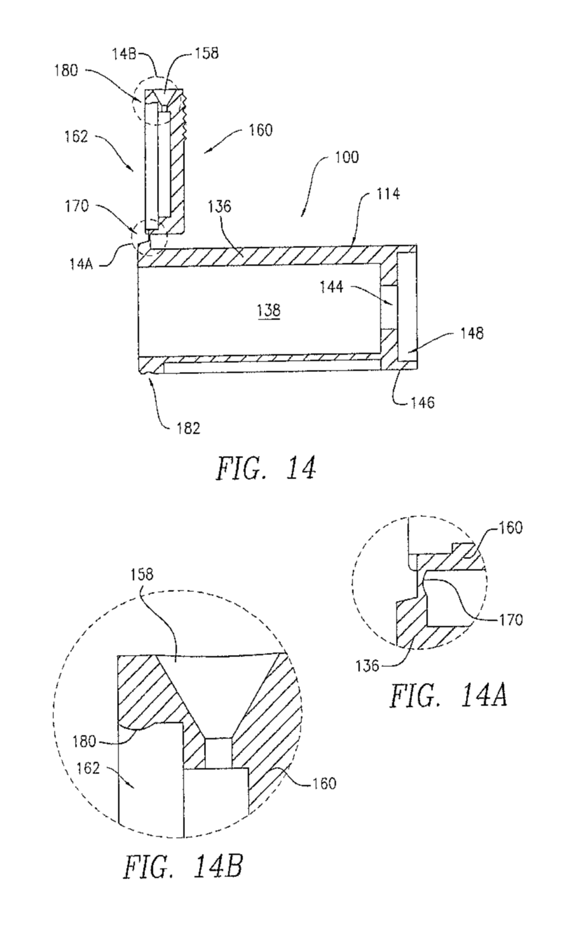

Turning now to FIGS. 13, 14, 14A, 14B and 15 there is illustrated a scent dispenser 100, which, unless indicated otherwise, includes the features mentioned above with respect to the scent dispenser 10. The scent dispenser 100, which may be made from a suitable plastic material, such as poly-vinyl chloride, includes a piston 114 having a piston body 136. The piston body 136 includes a hollow interior forming a cylindrical chamber 138 with a bottom opening 144 (see FIG. 14). The piston body 136 includes a bottom end 146 with a hollow opening forming a spring receiving bore 148 (see FIG. 14). The exterior of the piston 114 is formed with an L-shaped grove 152 having a vertical leg 154 and a horizontal leg 156 (see FIG. 13), which are sized and shaped similar to and function the same as the vertical leg 54 and the horizontal leg 56 of the scent dispenser 10 of FIGS. 1-12.

The scent dispenser 100 includes a hinged top 160, with a hollow interior 1 and a ventilation opening 158 formed in a side wall of the hollow interior 162. The hinged top 160 is connected to the piston body 136 by a hinge 170. FIG. 14A is a close up of the area 14A in FIG. 14. The hinged top 160 may be formed with a snap lock protrusion 180, which may engage a detent groove 182 on the exterior wall of the piston body 136 (see FIG. 14). FIG. 14B is a close up view of the area 14B in FIG. 14 showing the snap lock protrusion 180.

In operation, the scent dispenser 100 operates similarly to the scent dispenser 10 illustrated in FIGS. 1-12, with the exception of the hinged top 160 replacing the door 26 in function, and the ventilation opening 58 is formed in the hinged top 160 in fluid communication with the cylindrical chamber 138. The piston 114 is inserted into a piston cylinder (not shown in FIGS. 13, 14, 14A, 14B and 15) having a circular cylinder opening (not shown) to receive the piston 114 in relatively sealing engagement (although a seal such as an o-ring may also be used) and sucks in ambient air through the scent dispenser 100 cartridge (not shown in FIGS. 13, 14, 14A, 14B and 15) as the piston 114 is moved to a withdrawn position. The air is then pushed out the ventilation opening 158 when the piston 114 is moved in the opposite direction to an inserted position, again passing through the cartridge (not shown) on the way out.

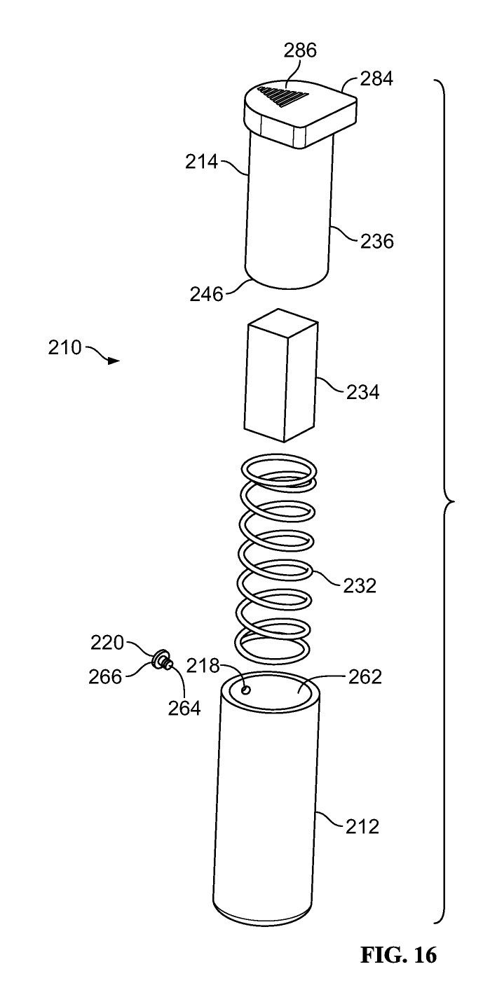

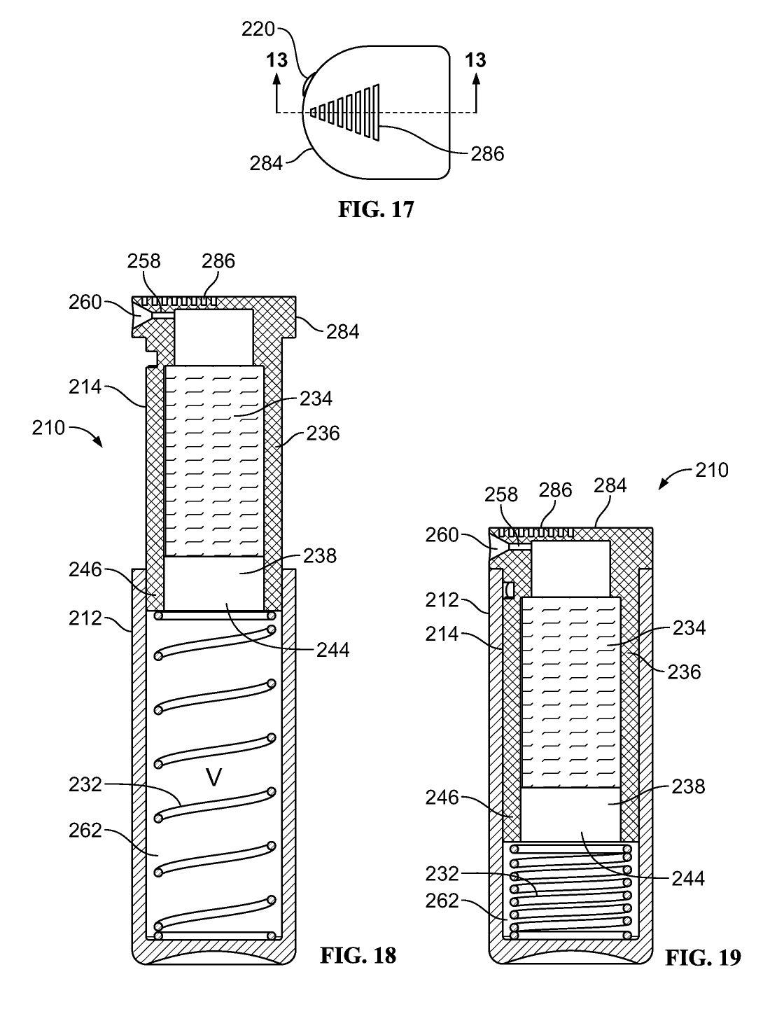

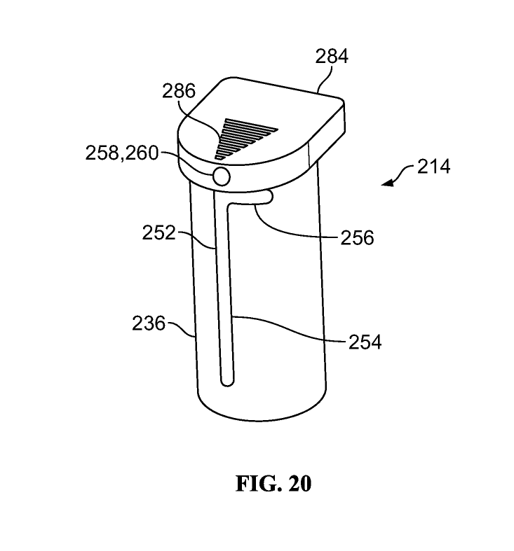

Another exemplary embodiment provides a scent dispenser 200 that is intended for single use and is shown in FIGS. 16-20. Unlike the first embodiment described hereinabove and which includes a removable door 26 fitted into a U-shaped flange 22 and groove 24, the scent dispenser 200 of the present exemplary embodiment does not include any of those features since it is not intended that the scent cell 234 be replaceable, as will be explained below. Generally, as shown most clearly in FIGS. 16 and 18-19, in this exemplary embodiment, the scent dispenser 200 includes a cylinder 212 having a cylinder chamber 262 sized and oriented to receive a piston 214 therein such that the piston 214 may move upward or downward within the cylinder 212 and cylinder chamber 262 between a fully extended position (e.g., FIG. 18) and a fully compressed position (e.g., FIG. 19). As also shown in FIGS. 16 and 18-19, the scent dispenser 200 includes a spring 232 and a scent cell 234 both of which will be discussed hereinafter in further detail. FIG. 20 provides a front perspective view of the piston 214 of the scent dispenser 200 showing an L-shaped groove 252 which is described in further detail below. Although it is not shown, this embodiment may also include a ring (e.g., similar to ring 30 in FIG. 1) for the attachment of the scent dispenser 200 to another object such as a lanyard or key ring.

As shown most clearly in FIGS. 16, 17 and 20, the scent dispenser 200 further includes a cover 284 which covers the top of piston 214 to form the top of the scent dispenser 200 when assembled and to retain the scent cell 234 in the piston 214. In some embodiments, the cover 284 may include surface features, such as ridges 286, to facilitate handling and operation of the scent dispenser 200 by a user. The cylinder 212 has a pin hole 218 to receive a lock pin 220, which can be seen in FIG. 16. The lock pin 220 has a small end 264 and a large end 266. The lock pin 220 is sized and oriented such that the small end 264 may extend through the pin hole 218 and into the L-shaped groove 252 of the piston 214. The large end 266 of the lock pin 220 is sized and shaped to prevent the lock pin 220 from being inserted all the way through the pin hole 218. In an alternative embodiment, the lock pin 220 may be an integral feature on the cylinder 212, extending into the cylinder chamber 262 and sized and oriented to fit and travel within the L-shaped groove 252 on the piston 214. As will be understood by persons of ordinary skill in the art, and although not shown per se in the figures, a further alternative embodiment may have the L-shaped groove 252 in the cylinder 212 and the lock pin 220 formed as an integral feature on the piston 214. In such an embodiment, the lock pin 220 on the piston 214 would extend radially from the piston 214 and be sized and oriented to fit and travel in the L-shaped groove 252 on the cylinder 212.

To understand the elements and function of the piston 214, FIGS. 18-19 should be reviewed together and in conjunction with FIG. 16. The piston 214 has a cylindrical piston body 236, which is attached to or integral with the cover 284. The piston body 236 is partially hollow and defines a piston chamber 238 arranged such that the scent cell 234 may be received entirely within the piston chamber 238. The piston body 236 defines an opening 244 at the bottom end 246 of the piston body 236 which allows insertion of the scent cell 234 into the piston chamber 238 during assembly of the scent dispenser 200. The opening 244 of the piston body 236 also provides fluid communication between the piston chamber 238 and the ambient environment of the piston body 236, i.e., into the cylinder chamber volume V (see FIG. 18) formed as the piston 214 is withdrawn from the cylinder 212.

Continuing to refer to FIGS. 18-19 in conjunction with FIG. 16, the piston body 236 defines a ventilation port 258 that provides fluid communication between the piston chamber 238 and the ambient environment of the scent dispenser 200. The ventilation port 258 is positioned such that the passage of air through the ventilation port 258 between the ambient environment and the piston chamber 238 is not impeded. Other forms of ventilation opening may be envisioned and employed by persons of ordinary skill in the relevant art.

In some embodiments, such as the illustrated embodiment, the ventilation port 258 is formed through or proximate the cover 284 as shown in FIGS. 18-20. In some embodiments, the ventilation port 258 has an effective diameter of 1 mm or less. In some embodiments, such as the illustrated embodiment, the ventilation port 258 is provided with a counterbore 260 that flares outward toward the ambient environment such as to reduce resistance of air flow into the piston chamber 238 through the ventilation port 258 and thereby aid the dispersal of scent passing out of the piston chamber 238 through the ventilation port 258. Air flow into and out of the piston chamber 238 occurs through the operation of the scent dispenser 10 as discussed hereinafter.

In the cross-sectional views provided by FIGS. 18-19, FIG. 18 shows the piston 212 in its fully extended position and FIG. 19 shows the piston 212 in its fully compressed position. As can be seen in FIGS. 18-19 in conjunction with FIG. 16, the cylinder 212 defines the cylindrical cylinder chamber 262 that is arranged to receive the piston body 236 such that the piston body 236 fits closely within the cylinder chamber 262 while allowing the piston body 236 to move readily upward or downward therein. The pin hole 218 penetrates the cylinder 212 such that the piston body 236 is exposed through the pin hole 218.

Referring still to FIGS. 18-19 in conjunction with FIG. 16, the spring 232 is arranged to fit within the cylinder chamber 262 in contact with bottom end 246 of the piston body 236. The spring 232 exerts an upward force on the piston 214 such that the piston 214 moves upward without being pulled (i.e., manually manipulated by a user). In some embodiments, such as the illustrated embodiment, the spring 232 is a helical compression spring.

With reference now to FIG. 20, a front perspective view is provided of the piston 214 showing the L-shaped groove 252 that is recessed into the piston body 236 and controls the movement of the piston 214 in the cylinder 212. The L-shaped groove 252 has a longitudinal leg 254 and a lateral leg 256. The L-shaped groove 252 is arranged such that its longitudinal leg 254 is oriented along the direction of travel of the piston 214 within the cylinder 212 and has a length equal to the distance over which the piston 214 is desired to travel. The L-shaped groove 252 is further arranged such that its lateral leg 56 is oriented to coincide with the pin hole 218 of the cylinder 212 when the piston body 236 is at its furthest limit of downward travel (i.e., the fully compressed position, FIG. 19) within the cylinder 212. The L-shaped groove 252 is sized and oriented to receive the lock pin 220 therein at all times.

Referring still to FIG. 20, in conjunction with FIG. 16, in the aforesaid arrangement of the pin hole 218, lock pin 220 and L-shaped groove 252, the lock pin 220 prevents the upward or downward movement of the piston 214 when the lock pin 220 resides in the lateral leg 256 of the L-shaped groove 252 and allows the upward or downward movement of the piston 214 when it resides in the longitudinal leg 254 of the L-shaped groove 52. The pin hole 218 and L-shaped groove 252 are arranged such that, when the piston body 236 is at its furthest limit of downward travel into the cylinder 212 (i.e., the fully compressed position, FIG. 19), the lock pin 220 may be moved from the longitudinal leg 254 to the lateral leg 256, or vice versa, by rotating the piston 214 and cylinder 212 relative to one another so that the lock pin 220 travels along the lateral leg 256 of the L-shaped groove 252, as desired.

The scent cell 234 is the same as described hereinabove in connection with other embodiments and includes a material, such as a substantially hydrophilic foam, that can absorb and/or adsorb a volatile scented substance and allows the ready passage of air through the scent cell 234. The scent cell 234 may be made entirely of such material or, as in some embodiments described above, it may include a substrate (not shown per se), such as a nonwoven fibrous material, that is covered, coated or otherwise combined with coating material, such as a substantially hydrophilic foam, that can absorb and/or adsorb a volatile scented substance and both the substrate and coating material allow the ready passage of air through the scent cell 234.

As with previous embodiments, the various elements of the scent dispenser 200 are arranged such that, when the scent dispenser 200 is fully assembled, the ventilation port 258 provides the only path by which air may move into or out of the scent dispenser 200. The fully-assembled scent dispenser 200 may be operated in the manner described herein with reference to FIGS. 16 and 18-19. Beginning in the assembled state and fully compressed position shown in FIG. 19, the cylinder 212 and piston 214 are rotated relative to one another to move the lock pin 220 along the lateral leg 256 of the L-shaped groove 252 such that its small end 264 enters the longitudinal leg 254 of the L-shaped groove 252. This action frees the piston 214 to travel upward toward its fully extended position (FIG. 18), with the motive force provided by the spring 232. Additionally, as the piston 214 travels upward, suction is created within the cylinder chamber 262, drawing air into the confined cylinder chamber volume V formed as the piston 214 advances upward in the cylinder chamber 262, through the ventilation port 258 and the piston chamber 238. As the air is drawn in, some or all of the air passes through the scent cell 34, carrying some of the volatile scented material into the cylinder chamber 262.

After the piston 214 has traveled upward for some distance, it may be pressed downward into the cylinder 212, such as by a user applying pressure on the cover 284 and piston 214 to counteract the spring force of spring 232. Such action causes the air in the cylinder chamber 262 to become compressed, forcing air to pass from the cylinder chamber 262 through the piston chamber 238, to be expelled through the ventilation port 258. Some or all of the air again passes through the scent cell 234, carrying away an additional amount of the volatile scented material which is expelled with the air through the ventilation port 258. The further the travel of the piston 214, the more air, and scent, is expelled. When the piston 214 is fully inserted into the cylinder 12 (i.e., its fully compressed position, FIG. 19), the cylinder 212 and piston 214 may again be rotated relative to one another to move the lock pin 220 into the lateral leg 256 of the L-shaped groove 252, preventing upward movement of the piston 214 that would otherwise be caused by the spring 232. Other mechanical or electromechanical mechanisms (not shown) may be utilized to move the piston 214 in the direction in which air is expelled from the cylinder chamber volume V returning through the scent cell 234 and out the ventilation opening 258.

It should be understood that the embodiments discussed herein are merely exemplary and that a person skilled in the relevant arts may make many variations and modifications thereto without departing from the spirit and scope of the invention. For example, in the illustrated embodiment, the cylinder 12, piston body 36 and cylinder chamber 62 substantially have the shape of a cylinder having a rectangular horizontal cross-section. In other embodiments, corresponding elements may be provided as cylinders having other horizontal cross-sectional shapes, such as a circular cross-section, an elliptical cross-section, or a non-rectangular polygonal cross-section. In embodiments having a circular cross-section, the ledge 16, slot 18 and lock button 20 may be replaced by an element, such as a screw, that is secured to the cylinder and protrudes into the interior of the cylinder such as to engage an L-shaped groove in the piston body that corresponds to the L-shaped groove 52 of the piston body 36. The piston body may then be released to move upward by rotating the piston, in the manner of opening a bayonet lock, such that the protruding element moves into the longitudinal leg of the L-shaped groove, allowing the piston to move upward. In some such embodiments, the rest of the operation of the scent dispenser would be the same as has been described above with respect to scent dispenser 10.

In some embodiments, the scent dispenser may be equipped with a seal, such as an o-ring seal on the outside of the body of the piston 14 near the bottom end 46 to facilitate the formation of the suction effect as the piston 14 moves within the cylinder 12 to form the confined cylinder chamber volume V, as discussed above. The cylinder 12 may also be of cylindrical shape, as opposed to the rectangular cylinder shape of the disclosed exemplary embodiment.

Further, the scent dispenser of the disclosed exemplary embodiment may serve as an odor or other toxicant-carrying material remover. The scent cell 34, instead of carrying material desired to be released into the atmosphere for the provision of some pleasing aroma or masking of some undesirable aroma, may capture and remove from the ambient environment undesired particulate or other materials causing an undesired aroma or other undesirable conditions in the ambient environment air. By acting as a particulate filter for particulate carried in the ambient atmosphere passed through the cartridge 34 according to the operation of the exemplary embodiment discussed in the present application, the scent dispenser 10 can also act to modify the ambient environment of the air around the dispenser 10.

It will be understood by those skilled in the art that an ambient environment modification mechanism is disclosed by way of example as a scent dispenser (which could also act as a filter) which may comprise the piston 14 and the piston cylinder 12, receiving the piston 14, and forming a cylinder chamber volume V when the piston 14 is moved from an inserted position in the piston cylinder 12 to a withdrawn position in the piston cylinder 12. The inserted position may be any point of insertion up to full insertion and the withdrawn position may be any point short of full withdrawal, which full withdrawal would break the air seal between ambient and the cylinder chamber volume V. The piston 14 may have a hollow interior 38 receiving an ambient air modification material holder, such as the cartridge 34, containing ambient air modification material, such as to emit material to generate a pleasing aroma in the ambient environment air or mask an undesired aroma in the ambient air or such as to filter particles adversely effecting the ambient environment air. The hollow interior 38 may be in fluid communication with the cylinder chamber volume V at a first end of the hollow interior 38. A ventilation opening such as ventilation port 58 may be in fluid communication with the hollow interior 28 at a second end of the hollow interior 38. A cylinder chamber volume V forming mechanism, such as spring 32 may move the piston 14 relative to the piston cylinder 12 from the inserted position to the withdrawn position.

It will be understood that the embodiments of the present invention described herein are merely exemplary and that a person skilled in the art may make many variations and modifications without departing from the spirit and scope of the invention. All such variations and modifications are intended to be included within the scope of the invention.

1. A scent dispenser, comprising:
a cylinder having a cylinder chamber, with an open end, a closed end, and a pin hole proximate the open end;
a lock pin having a small end sized and shaped to fit through the pin hole and extend into the cylinder chamber and a large end sized and shaped to retain the lock pin in the pin hole;
a piston having a cylindrical piston body which is partially hollow and sized and shaped to be inserted into the cylinder chamber through the open end thereof, wherein the piston is slideable, in a longitudinal direction, in the cylinder chamber, between a fully extended position in which the piston extends out of the cylinder chamber, and a fully compressed position in which the cylindrical piston body is fully received within the cylinder chamber; the cylindrical piston body having a bottom end, an upper end opposite the bottom end, and a piston chamber which is open at a bottom end of the piston body, wherein the piston chamber is sized and shaped to receive a scent cell therein through the open bottom end of the piston body; the cylindrical piston body having a ventilation port proximate its upper end and in fluid communication with the piston chamber for allowing air to pass into the piston chamber, contact a scent cell held therein, and pass out of the piston chamber and out of the scent dispenser; the cylindrical piston body having an L-shaped groove which is sized and oriented to receive the lock pin therein, the L-shaped groove having a longitudinal leg and a lateral leg, wherein the longitudinal leg is oriented along the longitudinal direction in which the piston slides within the cylinder chamber and has a length equal to a distance between the fully extended and fully compressed positions of the piston, and the lateral leg is oriented to coincide with the pin hole of the cylinder when the piston is in its fully compressed position in the cylinder chamber;
a spring which is positioned in the cylinder chamber, in contact with the closed end of the cylinder chamber and with the bottom end of the cylindrical piston body, wherein the spring urges the piston toward its fully extended position;
a cover which is attached to or integral with the piston at the upper end of the cylindrical piston body, whereby the cover covers the upper end of piston body and retains the scent cell in the piston chamber; wherein the cover includes surface features for facilitating handling and operation of the scent dispenser by a user.

2. The scent dispenser of claim 1, further comprising a scent cell which comprises:
a substrate comprising a nonwoven fibrous material and being capable of allowing air to flow readily through the scent cell;
a volatile scented substance; and,
optionally, a coating material capable of reversibly absorbing or adsorbing the volatile scented substance and capable of allowing air to flow readily through the scent cell, wherein the coating material comprises a substantially hydrophilic foam and covers or coats the substrate.

3. A scent cell comprising:
a substrate comprising a nonwoven fibrous material and being capable of allowing air to flow readily through the scent cell;
a volatile scented substance; and,
optionally, a coating material capable of reversibly absorbing or adsorbing the volatile scented substance and capable of allowing air to flow readily through the scent cell, wherein the coating material comprises a substantially hydrophilic foam and covers or coats the substrate.
